(19) United States
(12) Patent Application Publication
(10) Pub. No.: US 2023/0100793 A1
(43) Pub. Date: Mar. 30, 2023

(54) RAPID-TESTING MASK

(71) Applicants: Chih-Cheng CHIANG, Taichung (TW); Tzu-Hsiang TSENG, Taichung (TW)

(72) Inventors: Chih-Cheng CHIANG, Taichung (TW); Tzu-Hsiang TSENG, Taichung (TW)

(21) Appl. No.: 17/949,513

(22) Filed: Sep. 21, 2022

(30) Foreign Application Priority Data
Sep. 28, 2021 (TW) 110211390

(51) Int. Cl. A41D 13/11; A62B 27/00; A62B 18/08; A62B 23/02

(52) U.S. Cl. CPC A41D 13/11; A62B 27/00; A62B 18/08; A62B 23/025

(57) ABSTRACT

A rapid-testing mask is worn by a user, and includes a covering portion, a strap portion and a rapid-testing portion. The covering portion is provided to cover the mouth and the nose of the user. The covering portion includes an inner layer close to the user and an outer layer opposite to the user. The strap portion is connected to the covering portion and is worn on the user. The rapid-testing portion is arranged on the inner layer of the covering portion, and receives a gas exhaled from the mouth or the nose of the user to generate an indication signal according to the gas. The indication signal is a rapid-testing result of whether the user is suffering from diseases of pneumonia, influenza and COVID-19 (Coronavirus disease 2019), to monitor health status of the user in real time.

### FIELD OF THE INVENTION

The invention relates to a mask, in particular to a mask with a rapid-testing function.

### BACKGROUND OF THE INVENTION

Respiratory diseases, such as upper and lower respiratory tract infections and influenza, are symptoms of inflammation caused by viral, bacterial or other microbial infections of a patient. It may occur in people of all ages and be fatal when the disease is severe if the inflammation of influenza, COVID-19 (Coronavirus disease 2019) or pneumonia spreads.

Early detection and early treatment are the best way to prevent influenza, COVID-19 and pneumonia from worsening and being difficult to treat. To determine whether a suspected patient has symptoms of pneumonia, U.S. Pat. No. 10,073,097 discloses an in vitro method for diagnosing and treating pneumonia of the patient. The conventional pneumonia screening device needs to extract a biological sample of the patient. Therefore, the consumed screening time is long and the steps are more complicated. Usually, the in vitro method can only be carried out in a hospital, and it is difficult to have the function of early detection.

U.S. Patent No. US 2018/0256087A1 discloses another pneumonia screening device. The pneumonia screening device adopts portable design, which is convenient for real-time detection. However, the pneumonia screening device is a special appliance and needs to be purchased on purpose. As a result, the patient purchases and uses the device only when a disease condition has worsened significantly, so it is difficult to detect the symptoms of pneumonia at the first time and to receive early treatment.

### SUMMARY OF THE INVENTION

The main purpose of the invention is to solve the problem that the conventional screening device is difficult to achieve early detection.

In order to achieve the above purpose, the invention provides a rapid-testing detection mask worn by a user and includes a covering portion, a lacing portion and a rapid-testing portion, wherein the covering portion is provided to cover the mouth and the nose of the user. The covering portion includes an inner layer and an outer layer, the inner layer is close to the user, and the outer layer is away from the user. The strap portion is connected to the covering portion and is worn on the user. The rapid-testing portion is arranged on the inner layer of the covering portion, and receives a gas to generate an indication signal according to the gas exhaled from the mouth or the nose of the user.

Accordingly, the invention provides a rapid-testing mask, which is worn by users infected with upper and lower respiratory tract infections. When wearing the rapid-testing mask, once the disease condition of the user worsens to have pneumonia or the user is infected with influenza or COVID-19, the gas is received through the rapid-testing portion to generate the indication signal accordingly, to achieve early detection. The user is noticed at the first time, and the user can undergo further detection and treatment to achieve the purpose of early treatment.

### BRIEF DESCRIPTION OF THE DRAWINGS

FIG. 1 is a schematic diagram of an embodiment of the invention.

FIG. 2 is a schematic diagram from another angle of the embodiment of the invention.

FIG. 3 is a schematic diagram of using the embodiment of the invention.

FIG. 4 is a schematic diagram of another embodiment of the invention.

### DETAILED DESCRIPTION OF THE PREFERRED EMBODIMENTS

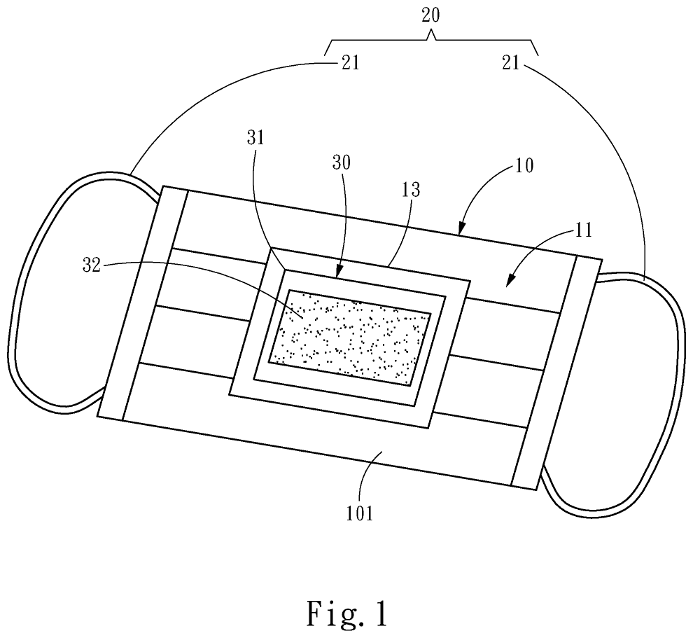

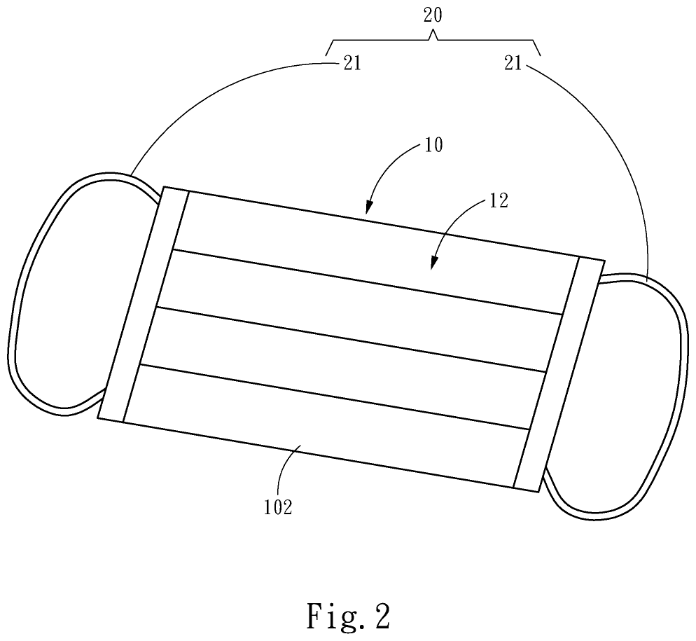

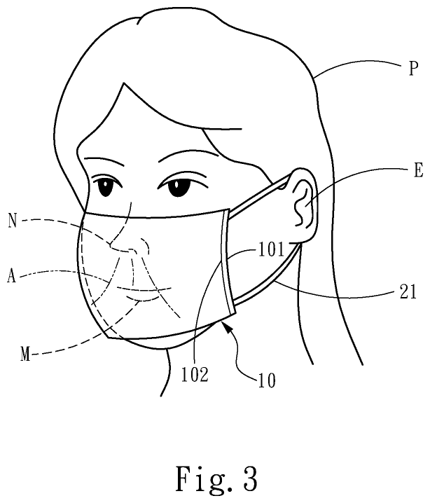

Referring to FIGS. 1, 2, and 3, the invention provides a rapid-testing mask which is worn by a user P and comprises a covering portion 10, a strap portion 20 and a rapid-testing portion 30, wherein the covering portion 10 is provided to cover the mouth M and the nose N of the user P. The covering portion 10 comprises an inner layer 101 and an outer layer 102. The inner layer 101 is close to the user P, and the outer layer 102 is opposite to the user P. In an embodiment, the covering portion 10 comprises a first non-woven fabric layer 11, a second non-woven fabric layer 12 and a filter layer (not shown) arranged between the first non-woven fabric layer 11 and the second non-woven fabric layer 12. The first non-woven fabric layer 11 is used as the inner layer 101, and the second non-woven fabric layer 12 is used as the outer layer 102.

The strap portion 20 is connected to the covering portion 10 and is worn on the user P. In an embodiment, the strap portion 20 comprises two cords 21 respectively looped and worn on ears E of the user P.

The rapid-testing portion 30 is arranged on the inner layer 101 of the covering portion 10. The rapid-testing portion 30 receives a gas A, and generates an indication signal according to the gas A. The gas A is exhaled from the mouth M or the nose N of the user P. The rapid-testing portion 30 comprises a base 31 and a material layer 32 that changes a color when absorbing a specific minimal substance (not shown). The material layer 32 is carried on the base 31 and absorbs the gas A. When the user P has one of pneumonia, influenza, or COVID-19, the gas A exhaled from the mouth M or the nose N of the user P contains the specific minimal substance. When the material layer 32 absorbs the gas A, that is, absorbs the specific minimal substance and changes the color, the material layer 32 that changed the color is used as the indication signal to warn that the user P may be infected with pneumonia, influenza, COVID-19, or the like. In other embodiments, the material layer 32 appears in the form of one line, two lines or no line to indicate whether the user has been infected or not.

In an embodiment, the covering portion 10 further comprises an adhesive layer 13. The adhesive layer 13 is arranged on the inner layer 101 to adhere the base 31 of the rapid-testing portion 30 to the inner layer 101. The rapid-testing portion 30 is fixed through the adhesive layer 13, so that the gas A exhaled from the mouth M or the nose N of the user P is received by the rapid-testing portion 30.

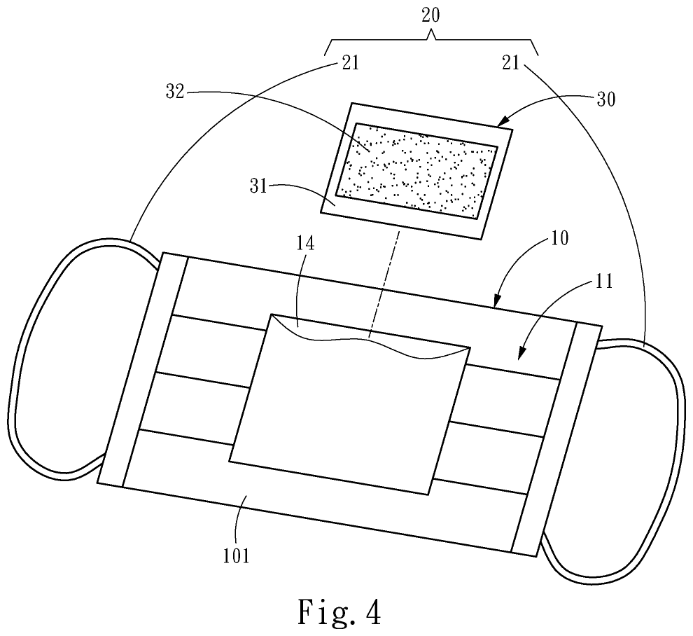

Referring to FIG. 4, in another embodiment, the covering portion 10 further comprises a pocket 14, and the pocket 14 is arranged on the inner layer 101 and to accommodate the base 31 of the rapid-testing portion 30. In this embodiment, in addition to fixing the rapid-testing portion 30, the pocket 14 also protects the material layer 32 on the base 31 to prevent the material layer 32 from directly contacting with a human body and problem of peeling off.

As mentioned above, compared with conventional technique, the invention has following advantages:

1. The rapid-testing portion is built with the mask, which monitors whether the user has pneumonia, influenza and (COVID-19) at any time, so as to achieve the purpose of early detection and early treatment.

2. Through the rapid-testing portion (the material layer), appears in the form of changing the color, one line, two lines, or no line as the indication signal, to clearly know whether there is a risk of infection.

3. The rapid-testing portion is fixed through the adhesive layer or the pocket, so that the structure is simple and the cost is low.

4. In addition to provide the function of preventing disease infection in the mask, the rapid-testing portion is also used to detect whether the user is infected with pneumonia, influenza and COVID-19, to ensure the health of the user.

What is claimed is:

1. A rapid-testing mask, worn by a user, comprising:
a covering portion, provided to cover a mouth and a nose of the user and comprises an inner layer close to the user and an outer layer opposite to the user;
a strap portion, connected to the covering portion and worn on the user; and
a rapid-testing portion, arranged on the inner layer of the covering portion, and receives a gas exhaled from the mouth or the nose of the user to generate an indication signal according to the gas.

2. The rapid-testing mask according to claim 1, wherein the rapid-testing portion comprises a base and a material layer that changes a color when absorbing a specific minimal substance, and the material layer is carried on the base and absorbs the gas.

3. The rapid-testing mask according to claim 2, wherein the covering portion comprises an adhesive layer arranged on the inner layer to adhere the base of the rapid-testing portion to the inner layer.

4. The rapid-testing mask according to claim 2, wherein the covering portion comprises a pocket arranged on the inner layer to accommodate the base of the rapid-testing portion.

5. The rapid-testing mask according to claim 1, wherein the covering portion comprises a first non-woven fabric layer, a second non-woven fabric layer and a filter layer arranged between the first non-woven fabric layer and the second non-woven fabric layer.

6. The rapid-testing mask according to claim 1, wherein the strap portion comprises two cords respectively looped and worn on ears of the user.
